(19) United States
(12) Patent Application Publication
(10) Pub. No.: US 2009/0221625 A1
(43) Pub. Date: Sep. 3, 2009

(54) COSMECEUTICAL COMPOSITION

(76) Inventors: Stefan Hirsch, Lorrach (DE); Katrin Kriwet, Grenzach-Wyhlen (DE); Dorothea Ledergerber, Inzlingen (DE); Friedrich Karl Mayer, Oberwil (CH); Nabila Sekkat, Basel (CH); Kurt Schmidt, Long Valley, NJ (US); Wanda Richard, Ada, MI (US)

Correspondence Address: Gerber Products Company, 12 Vreeland Road, 2nd Floor, Box 697, Florham Park, NJ, 07932, US

(21) Appl. No.: 11/912,372

(22) PCT Filed: Apr. 27, 2006
(86) PCT No.: PCT/EP06/03930

§ 371 Date: Jul. 8, 2008

(30) Foreign Application Priority Data
Apr. 29, 2005 (GB) 0508827.3

(51) Int. Cl. A61K 31/4745; A61K 31/08; A61K 31/20; A61P 17/00; A61P 17/16

(52) U.S. Cl. 514/291; 514/723; 514/558

(57) ABSTRACT

Topical compositions comprising: —a physiologically acceptable alkanediol, ether diol or diether alcohol containing up to (8) carbon atoms; —water; and—optionally an unsaturated fatty alcohol; and optionally further conventional excipients, for use as a cosmeceutical, in particular for use in the repair or maintenance of skin barrier function. They are indicated for use in e.g. moisturizing skin, nail and mucosa and, when an optional pharmaceutically active agent is present, additionally in the treatment of various skin, nail and mucosal diseases. Also disclosed are compositions suitable for topical application to infants and babies for treating dry skin, and soothing skin irritated from diaper rash and scrapes.

The invention relates to pharmaceutical and/or cosmeceutical compositions, for use in particular on skin.

EP 0 786986 B1 claims the use of an unsaturated fatty alcohol to stabilise a macrolide active agent in a pharmaceutical composition. Disclosed therein are topical pharmaceutical compositions in the form of an emulsion comprising

a macrolide compound of the FK506 class;

a physiologically acceptable alkanediol, ether diol or diether alcohol containing up to 8 carbon atoms as solvent for the compound of the FK506 class; and

water;

characterised in that it further comprises an unsaturated fatty alcohol; and optionally further excipients.

It has now been found that, surprisingly, even in the absence of the pharmaceutically active macrolide compound, the above composition vehicle displays excellent cosmeceutical properties, as attested by its repair or maintaining activity on skin barrier function.

It may therefore find use as a so-named “cosmeceutical [Nature 424 (2003) 990-991] as such, or optionally together with a further pharmaceutically active agent.

Specifically, the invention concerns a topical composition comprising:

a physiologically acceptable alkanediol, ether diol or diether alcohol containing up to 8 carbon atoms;

water; and

optionally an unsaturated fatty alcohol; and

optionally further conventional excipients;
or use as a cosmeceutical, in particular, for use in the repair or maintenance of skin barrier function, hereinafter briefly named “the composition of the invention”.

The invention further concerns the use as a cosmeceutical, in particular, the use in the repair or maintenance of skin barrier function, of a topical composition as defined above, and optionally comprising a pharmaceutically active agent, including e.g. a compound of the FK506 class, hereinafter briefly named “the use of the invention”.

A compound of the FK506 class is e.g. FK506 (tacrolimus) as such or a compound which has the same basic structure as FK506 and which has at least one of the biological properties, for example, immunosuppressant properties, of FK506, such as ascomycin; it preferably is 33-epichloro-33-desoxyascomycin of formula I

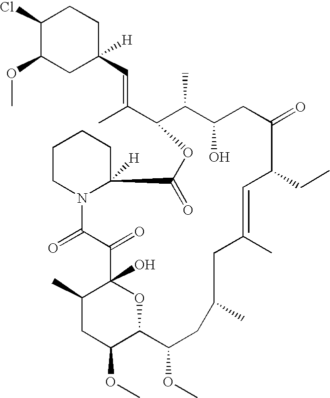

disclosed e.g. as Example 66a in EP 427680, and known under the generic name pimecrolimus (Elide^{R}).

Elide^{R} is marketed as a 1% w/w cream emulsion in a vehicle comprising the following excipients (w/w) in addition to the active agent:

| | | |
|---|---|---|
| physiologically acceptable alkanediol solvent: | propyleneglycol | 5% |
| water: | water, purified: | ad 100% |
| unsaturated fatty alcohol: | oleyl alcohol | 10% |

further excipients:

| | | |
|---|---|---|
| further liquid oil: | medium chain triglycerides | 15%; |
| thickening agent (and emulsifier): | cetyl alcohol | 4%; |
| thickening agent (and emulsifier): | stearyl alcohol | 4%; |
| emulsifier: | sodium cetylstearyl sulfate | 1%; |
| emulsifier: | glycerine mono-/distearate | 2%; |
| preserving agent: | benzyl alcohol | 1%; and |
| buffer: | citric acid 0.05/sodium hydroxide | 0.02%; |

as reflected in i.a. Example 14 of EP 0 786986 B1 (with the amount of active agent 1% in place of 0.3% and preserving agent benzyl alcohol in place of Methyl Paraben 0.07%/Propyl Paraben 0.03%).

The composition of the invention preferably is in the form of an emulsion. It preferably is free of petrolatum, paraffin and vaseline.

“%” as used herein means percent on a weight basis (w/w).

“Topical” includes, in addition to skin, also mucosa and nail.

The physiologically acceptable alkanediol, ether diol or diether alcohol preferably contains 3-8, and more preferably 3-6, carbon atoms. Examples of physiologically acceptable alkanediol components are propyleneglycol (1,2-propanediol), butyleneglycol, 2-ethyl-1,3-hexanediol, hexyleneglycol (2-methyl-2,4-pentanediol) and the like. Examples of ether diols are dipropyleneglycol, diethyleneglycol and the like. Examples of diether alcohols are diethyleneglycol monoethylether and the like. Preferably that component is propyleneglycol or hexyleneglycol, especially propyleneglycol. It preferably is present in an amount of about 5% to about 50%, more preferably about 5% to about 20% and even more preferably about 5% to about 10% of the total weight of the composition.

The oil phase of the composition may comprise about 20% to about 80%, more preferably about 25% to about 75% and even more preferably about 35% to about 65% of the composition. The composition preferably is an oil-in-water emulsion. The oil-in-water emulsion may e.g. be in the form of an emulsion gel (in which case the continuous aqueous phase may be thickened using a polymeric thickener), or in the form of a cream.

The optional unsaturated fatty alcohol forms part of the oil phase of the composition and is preferably a lanolin alcohol or a C₁₆₋₁₈ fatty alcohol; more preferably oleyl alcohol, or elaidic alcohol, although oleyl alcohol is particularly preferred. The composition preferably contains about 2% to about 10% and even more preferably about 5% to about 10%.

The oil phase also may contain further liquid oils and thickening agents conventionally used in topical compositions.

Suitable further liquid oils include medium chain triglycerides obtained from fractionated vegetable oils, such as capryl/caprinic acid triglycerides. One example of such a triglyceride is commercially available under the trade name Miglyol 812^{R} (which has a molecular weight of about 520, a n_{D}²⁰ of about 1.448 to 1.450 and a viscosity of 0.28 to 0.32 Pa·s). Another example is Captex^{R} 355 (Abitec Corp., Columbus, Ohio), derived from coconut oil fatty acids, having a specific gravity of 0.92-0.96 at 25° C. and a viscosity of 20-25 cP at 25° C. (Brookfield). Still another suitable liquid oil is sunflower seed oil, which may be commercially obtained under the trade name Lipovol® SUN from Lipo Chemicals Inc. (Paterson, N.J.). Also suitable are silicon oils, e.g., dimethicone (i.e. polydimethylsiloxane), preferably of medium viscosity, preferably about 50 cStk, e.g., Dow Corning 200® Fluid, 50 Cst. Such liquid oils may be used alone or in mixtures.

The total amount of liquid oil may comprise about 5% to about 60% of the composition and preferably about 5% to about 30%, e.g., about 5% to about 15%.

Suitable thickening agents include conventional stiffeners such as cetyl alcohol, cetostearyl alcohol, stearyl alcohol, hydrogenated castor oil (Cutina HR^{R}), Yellow wax, White wax, cetyl ester wax, emulsifying wax, microcrystalline wax, and the like. Preferably the thickening agent forms about 2% to about 30% of the composition and more preferably about 2% to about 10%.

The composition may also include suitable emulsifiers as is usual in emulsion compositions. Such emulsifiers are described in standard texts such as Fiedler, H. P., Lexikon der Hilfsstoffe für Pharmazie, Kosmetik und angrenzende Gebiete (1989), Editio Cantor, D-7960 Aulendorf, Germany and Handbook of Pharmaceutical Excipients (1986), A Joint Publication of the American Pharmaceutical Association, Washington D.C., USA and the Pharmaceutical Society of Great Britain, London, UK. Examples of suitable emulsifiers include:
(a) propyleneglycol mono- and di-fatty acid esters such as propyleneglycol dicaprylate (which is commercially available under the trademark Miglyol 840^{R}), propyleneglycol dilaurate, propyleneglycol hydroxystearate, propyleneglycol isostearate, propyleneglycol laurate, propyleneglycol ricinoleate, and propyleneglycol stearate;
(b) polyoxyethylene sorbitan fatty acid esters, such as mono- and tri-lauryl, palmityl, stearyl and oleyl esters. Examples of commercially available esters are polysorbates, such as those available under the trademark Tween^{R} (see Fiedler, pages 1300 to 1304) and particularly Tween 60^{R} (polysorbate 60) [polyoxyethylene(20) sorbitan monostearate] and Tween 80^{R} (polysorbate 80) [polyoxyethylene(20) sorbitan monooleate];
(c) polyoxyethylene fatty acid esters, for example polyoxyethylene stearic acid esters of the type known and commercially available under the trademark Myrj^{R} (see Fiedler, pages 834 and 835) and in particular Myrj 52^{R} (which has a D²⁵ of about 1.1, a melting point of about 40 to 44° C., and a HLB value of about 16.9);
(d) polyoxvethylene-polyoxypropylene co-polymers and block co-polymers such as those known and commercially available under the trademarks Pluronic^{R}, Emkalyx^{R} and Poloxamer^{R} (see Fiedler, page 959) and in particular Pluronic F68^{R} (which has a melting point of about 52° C. and a molecular weight of about 6800 to 8975) and Poloxamer 188^{R};
(e) dioctylsulfosuccinate or di-[2-ethylhexyl]-succinate;
(f) phospholipids and in particular lecithins (see Fiedler, pages 943 and 944);
g) salts of fatty alcohol sulphates such as sodium lauryl sulfate and sodium cetylstearyl sulphate;
(h) sorbitan fatty acid esters such as sorbitan monostearate and sorbitan monooleate which are commercially available under the trademarks Arlacel 60^{R} (which has an HLB of about 4.7 and a melting point of about 53° C.) and Span 80^{R} (which has a D²⁵ of about 1, an HLB of about 4.3 and a viscosity of about 950 to 100 cP);
(i) glycerine mono-/distearate with is available under the trademark Imwitor^{R} (see Fiedler, page 645) and particularly Imwitor 960^{R};
(j) esters of polyethyleneglycol glycerol ethers, that have at least one free hydroxyl group, and aliphatic C₆-C₂₂ carboxylic acids. Examples include PEG-20 glycerine monostearate;
(k) reaction products of a natural or hydrogenated castor oil and ethyleneoxide and of which examples are available under the trademarks Cremophor^{R} such as Cremophor RH 40^{R} (having a saponification number of about 50 to 60, an acid number of<1, an n_{D}⁶⁰ of about 1.453 to 1.457 and an HLB value of about 14 to 16), Cremophor RH 60^{R} (having a saponification number of about 40 to 50, an acid number of<1, an n_{D}²⁵ of about 1.453 to 1.457 and an HLB value of about 15 to 17) and Cremophor EL^{R} (having a saponification number of about 65 to 70, an acid number of about 2, an n_{D}²⁵ of about 1.471 and a molecular weight of about 1630). Also suitable are various tensides available under the trademarks Nikkol^{R}, Emulgin^{R}, Mapeg^{R} and Incrocas^{R} (see Fiedler);
(l) stearic acid;
(m) oil and wax based emulsifiers such as cetyl alcohol and emulsifying wax;
(n) polyoxyethylene glycerides such as those available under the trademark Labrafil M2130 CS^{R} (see Fiedler, page 707);
(o) polyoxyethylene alkyl ethers such as polyoxyethylene stearyl ether, polyoxyethylene oleyl ether and polyoxyethylene cetyl ether which are available under the Brij^{R} and Cetomacrogol^{R} series trademarks (see Fiedler, pages 222 to 224 and 284);
(p) glycerine sorbitan fatty acid esters such as that available under the trademark Arlacel 481^{R} (which has a molecular weight of about 630 and an HLB value of about 4.5);
(q) sodium stearoyl dilactate, in particular having a hydrophilic-lipophilic balance (HLB) of about 7 to about 10, e.g., Capmul S18L^{R} (Abitec Corp., Columbus, Ohio).
   and
(r) mixtures thereof.

Preferably the emulsifier is selected from polyethyleneglycol (20) glycerine monostearate, sorbitan monostearate (Arlacel 60^{R}), sorbitan monooleate (Span 60^{R}), polysorbate 60 (Tween 60^{R}), polysorbate 80 (Tween 80^{R}), glycerine monostearate (Imwitor 960^{R}), stearic acid, cetyl alcohol, wool wax derivatives and alcohols and Labrafil M2130 CS^{R} and mixtures thereof. If the emulsion is a water-in-oil emulsion, the emulsifier selected preferably has a HLB value of 10 to 15. If the emulsion is an oil-in-water emulsion, the emulsifier selected preferably has a HLB value of 4 to 8. Preferably the emulsifiers are present in an amount of about 1% to about 30% and preferably from about 10% to about 25%.

Gelling agents may also be added to provide a gelled emulsion. Suitable gelling agents are carbomers (polyacrylic acid derivatives); such as those available under the trademark Carbopol^{R} (see Fiedler, pages 254 to 256). Carbopol 974^{R} and Carbopol 1342^{R} are preferred. The gelling agents are preferably present in an amount of about 0.2% to about 2%; more preferably less than about 1%.

The composition may also include preserving agents and anti-oxidants such as benzyl alcohol, butyl-hydroxytoluene, ascorbyl palmitate, sodium pyrosulphite, butyl hydroxy anisole, propyl p-hydroxybenzoate (available commercially, for example, under the trademark Paraben^{R}), methyl or propyl p-hydroxybenzoate (available commercially, e.g. as Paraben^{R}, such as Methylparaben^{R} or Propylparaben^{R}), sorbic acid and tocopherol. The preserving agents and anti-oxidants serve to prevent bacterial growth, and are preferably present in an amount of about 0.01% to about 2.5%. pH modifying agents may be included to bring the pH of the composition to between about 4 and about 6 or by adding a pharmaceutically acceptable buffer system. A pH of between 4 and 6, preferably about 5.5, is desirable to avoid skin irritation.

The composition of the invention may optionally comprise further conventional excipients, such as plasticizers, humectants (e.g. glycerol, propane-1,2-diol, polypropylene glycol and other polyhydric alcohols), free radical scavengers, viscosity-adjusting agents, dyes and colorants, e.g. as described in H. P. Fiedler, “Lexikon der Hilfsstoffe für Pharmazie, Kosmetik und angrenzende Gebiete”, Editio Cantor Verlag Aulendorf, Aulendorf, 5th Edition (2002), as well as fragrance.

The aqueous phase of the composition may comprise about 20% to about 80%, more preferably about 25% to about 75% and even more preferably about 35% to about 65% of the composition. The aqueous phase is preferably in the form of purified water.

If present, the pharmaceutically active agent, e.g. pimecrolimus, preferably is present in the composition in an amount of from about 0.01% to about 10% and more preferably from about 0.1% to about 1%. Preferably the active agent, if present, and the unsaturated fatty alcohol are in a weight ratio of from about 1:1000 to about 5: 1; preferably from about 1:100 to about 1:5.

A preferred group of compositions of the invention comprises:
propyleneglycol; preferably from about 1% to about 20%, especially about 5%;
water; preferably from about 25% to about 75%, especially from about 50% to about 60%; and
optionally oleyl alcohol; preferably from about 5% to about 20%, especially about 10%; and
further excipients, namely
   medium-chain triglycerides; preferably from about 5% to about 50%, especially about 15%; preferably Miglyol 812^{R};
   cetyl alcohol; preferably from about 2% to about 10%, especially about 4%;
   stearyl alcohol; preferably from about 2% to about 10%, especially about 4%;
   sodium cetylstearyl sulfate or polysorbate 60, preferably from about 0.5% to about 6%, especially about 1%;
   glycerine mono/distearate or sorbitan monostearate; preferably from about 1% to about 5%, especially about 2%;
   benzyl alcohol or methyl- and/or propylparaben; preferably from about 0.05% to about 2%, especially about 1%; and
   optionally citric acid/sodium hydroxide (buffer); preferably to bring the pH to between about 4 and about 6, especially to about 5.5;
      and optionally further conventional excipients.

Preferably the composition of the invention is devoid of paraffin, vaseline and petrolatum.

The composition of the invention is effective independently of the condition of the skin or mucosa, is well tolerated, stable and has particularly interesting solubilization and penetration properties for both lipophilic and hydrophilic pharmaceutically active agents.

It retains and improves on the beneficial penetration properties of more complex or inhomogenous formulations such as water- or hydrocarbon-based emulsions, while being particularly convenient in terms of ease of administration and subject compliance. It has the advantage of consisting of few components, is straightforward to prepare and well-tolerated on human skin and mucosa. It is non-oily, surprisingly non-irritating, and has pleasing cosmetic appeal, while being soft, easy to spread, rapidly penetrating into and absorbed by the skin, nail or mucosa, and providing a hydrating effect. It is particularly indicated for use on dry or sensitive skin, e.g. for maintenance or repair of normal skin barrier function when the subject is moving from a humid to a dry environment or country area.

In particular, the compositions are useful as infant and baby lotions, i.e. in treating dry skin, and in soothing skin irritated from diaper rash and scrapes. Such compositions may comprise, for example,
a physiologically acceptable alkanediol, ether diol or diether alcohol containing up to 8 carbon atoms (e.g., about 1-10 wt. %);
water; and
optionally an unsaturated fatty alcohol; and
   optionally further conventional excipients, such as:
a liquid oil component selected from one or more of dimethicone, sunflower oil and capric/caprylic triglycerides, and
an emulsifier selected from one or more of glyceryl monostearate, cetyl alcohol, stearyl alcohol, and sodium stearyl lactylate.

Further examples of compositions of the invention are topical compositions as defined above, comprising at least one pharmaceutically active compound, provided that said at least one pharmaceutically active compound is other than a pharmaceutically active macrolide compound of the FK 506 class.

Oil or extract of Calendula officinalis, i.e. the common marigold, may usefully be included as such an anti-inflammatory agent in the compositions herein described. Calendula Phytexcell®, an extract marketed by Croda (Edison, N.J.) is a suitable product for use in the present compositions.

Another such pharmaceutically active agent having useful anti-oxidant, anti-inflammatory properties is Vitamin E, which is preferably employed in the alpha or gamma form, most preferably in the alpha form. It is particularly useful to employ tocopheryl acetate, and especially alpha tocopheryl acetate (alpha-TAc), in the compositions of the invention, since as an oil it contributes to the barrier, protective function of the composition. The Vitamin E used in the invention may be either in the synthesized (i.e. d,l) form or in the natural (i.e. d) form.

In particular, it has been found that topical compositions comprising superior anti-inflammatory and anti-oxidative effects can be prepared comprising the combination of Calendula extract and alpha-TAc, preferably in an amount of about 0.005 to 0.05 wt. % Calendula extract and about 0.01 to about 0.1 wt. % alpha-TAC e.g., about 0.01 wt. % Calendula extract and about 0.05% alpha-TAc (amounts based on the total composition). Such compositions are preferably prepared as oil-in-water emulsions.

Another active agent having anti-oxidant, anti-inflammatory, wound-healing and other properties when applied topically is Aloe, e.g., Aloe barbadensis, also known as Aloe vera. Also suitable as active agents are chamomile, Vitamin A, and Vitamin D.

The compositions of the invention are suitable for use as topical compositions for infants and babies. They serve a useful skin barrier function as well as protect against moisture loss from the skin. Advantageously, the compositions moisturize with similar oils as are found in the infant's/baby's own skin. The compositions are therefore indicated for use in treating dry skin, diaper rashes and scrapes. The compositions may also include one or more active pharmaceutical agents other than a pharmaceutically active macrolide compound of the FK 506 class (e.g., Calendula extract and/or Vitamin E) for the greater therapeutic benefits to be derived therefrom.

An example of a topical composition for infants and babies may comprise:

| Amount | | |
|---|---|---|
| (wt. % based on | | |
| total composition) | Excipient | Function |
| 50-70 | purified water | diluent |
| 5-10 | dimethicone | skin protectant |
| 1-5 | sunflower oil | emollient |
| 1-15 | capric/caprylic triglycerides | emollient, Moisturizer |
| 1-10 | propylene glycol | stabilizing agent |
| 1-5 | glyceryl monostearate, SE | emulsifier |
| 2-10 | cetyl alcohol | co-emulsifier, emollient |
| 1-5 | stearyl alcohol | co-emulsifier, emollient |
| 1-5 | sodium stearyl lactylate | emulsifier |
| q.s. | e.g., sodium hydroxide, to | pH adjuster |
| | bring pH to 5.5 | |

The above composition may also optionally comprise active agents having anti-oxidant, anti-inflammatory properties, such as e.g. tocopheryl acetate (e.g., 0.01-0.1 wt. %) and Calendula extract (e.g., 0.005-0.05 wt. %).

Of course, in providing a skin barrier, or moisture-protecting function such compositions may be topically administered to a subject in need thereof, not limited to infants and babies. Thus such compositions in themselves are useful as “cosmeceuticals” (i.e. cosmetic products having medicament or drug-like benefits) in treating a mammalian subject (esp. human) in need of repair/maintenance of skin barrier function. The compositions are administered by single or repeated topical application to the area in need of moisture protection.

The utility of the composition of the invention in repair/maintenance of skin barrier function, and the use of the invention, can be observed in vivo in human or animal studies, such as e.g. by analyzing skin redness and (immuno)histological status of skin biopsies, and/or by measuring the decrease in transepidermal water loss (TEWL), a biophysical marker of skin barrier function for e.g. emollient and/or moisturizing properties, e.g. in human subjects or minipigs with induced inflamed skin receiving composition of the invention, e.g. using as comparators some of the single components of the composition of the invention defined above, such as oleyl alcohol, medium chain triglycerides and propyleneglycol.

In vivo animal testing is effected as follows:

Inflamed minipig skin: at four test sites on the dorsolateral trunk of animals an irritant contact dermatitis (ICD) is induced with 5% sodium lauryl sulfate (SLS). SLS (Fluka) is dissolved in water (500 mg in 3 ml) and mixed with 7 mg Eucerinum anhydricum (Beiersdorf, Vienna, Austria) 3:7 v/w. This formulation is applied under occlusion in self-made chambers to the test sites for 48 hours. The chambers are rectangular 4×4 cm frames of flexible 2 mm thick plastic, which are attached to the test sites, filled with the SLS formulation and covered with a tin foil, and finally fixed with Tegaderm^{R} (3M). ICD is assessed by clinical examination, measurement of redness with reflectometry (CR 200, Minolta) and by determination of TEWL with the Tewameter TM 210^{R} (Courage+Khazaka) 2 hours after the removal of the 48-hours patch.

Composition of the invention and single components are then applied on the treated sites. An untreated site is demarcated and taken as control.

Measurements of reflectometry and TEWL are performed in parallel on treated and untreated sites. TEWL measurements are effected with the Tewameter at t₀ (before product application), t₁₈₀ (180 minutes after application) and t₃₆₀ (360 minutes after application). The mean values obtained at each timepoint for transepidermal water loss on the treated area and on the control area is calculated for each animal, and appropriate statistical analysis is effected.

The utility of the composition of the invention, and the use of the invention, can also be observed in vivo in standard clinical tests, such as e.g. by measuring the decrease in TEWL in subjects receiving composition of the invention, e.g. using as comparators standard cosmeceutical formulations such as Nivea® Soft Cream (Beiersdorf), Cold Cream Naturel (La Roche Posay) or Oilatum® Lotion (Stiefel); e.g. as follows:

### a) Evaporimetry (TEWL):

Twenty female volunteers are selected, of all races and skin types, in the age range 18-60 years, who have been approved in a medical screening facility according to the specific inclusion and exclusion criteria adopted; the subjects are submitted to an interview and to a dermatological examination; they remain at rest in a climatized room (under controlled conditions of temperature and relative humidity) for 30 minutes before and throughout the test. The areas for application of composition and control formulation are demarcated on the anterior area of the arms following a randomized distribution.

Measurements are effected with a Tewameter at t₀ (before product application), t₁₈₀ (180 minutes after application) and t₃₆₀ (360 minutes after application). The mean values obtained at each timepoint for transepidermal water loss on the treated area and on the control area is calculated for each subject of the group, and appropriate statistical analysis is effected.

Evaporimetry Results. The composition of the invention maintained transepidermal water loss at the time points assessed.

### b) Moisturisation:

This is effected with twenty female volunteers under conditions as under a) above except that the areas for application of composition and control are demarcated on the anterior area of the legs in place of the arms, and moisture measurements are effected at to and at 1, 2, 3 and 6 hours after application.

Skin moisture (corneometry) Results. The composition of the invention promoted improved skin moisturization in relation to one of the comparators at all time points assessed and another comparator at T₃ hours.

In another in vivo clinical study, 16 patients with eczema were treated for 2 weeks twice daily with the specific 1% w/w cream emulsion vehicle for Elidel^{R} defined above. Of these, only 5 patients (31%) felt that no control of their eczema resulted, while 11 patients (69%) experienced some control of their eczema, indicative of restoration/repair of skin barrier function.

Satisfactory results are obtained in larger mammals, for examples humans, with topical application over the area to be treated of a concentration of active agent, if present, of 0.01% to 10%, preferably 0.1% to 3%, once or several times a day, e.g. 2 to 5 times a day.

In general, the composition of the invention may be applied to areas of skin as small as 1 cm² to as large as 1 m². Suitable skin loadings of pharmaceutically active agent, if present, fall within the range of 0.1 mg/cm² to 1 mg/cm².

The composition of the invention is well tolerated on skin and mucosa and good skin penetration and permeation rates may be achieved.

The invention further provides a method for repairing or maintaining skin barrier function comprising administering a composition of the invention to a subject in need thereof.

Still further, it provides the use of a composition of the invention in the preparation of a cosmeceutical composition.

The invention thus provides for the use as a cosmeceutical of a composition of the invention as defined above, e.g., of a composition additionally comprising a pharmaceutically active agent which is a macrolide of the FK506 class, such as pimecrolimus.

When it comprises a macrolide compound as defined above, such cosmeceutical composition is indicated for use also in the treatment of inflammatory and hyperproliferative skin diseases and of cutaneous manifestations of immunologically-mediated diseases. The terms “skin” and “cutaneous” should be understood broadly as comprising also diseases of e.g. nail or mucosa. Examples of immunologically-mediated diseases include alopecia areata, psoriasis, atopic dermatitis, contact dermatitis and further eczematous dermatitises, seborrhoeic dermatitis, lichen planus, pemphigus, bullous pemphigoid, epidermolysis bullosa, urticaria, angioedemas, vasculitides, erythemas, cutaneous eosinophilias, and lupus erythematous. Examples of skin diseases include dermatomyositis, leukoderma vulgaris, ichthyosis vulgaris, photoallergic sensitivity, cutaneous T cell lymphoma, acne, autoimmune diseases such as chronic rheumatoid arthritis, scleroderma and the like.

The composition of the invention may be prepared in conventional manner by working up the components into a cosmeceutical composition, e.g. by dissolving or mixing the non-watery component excipients into or with each other, and then adding the resultant oil phase to the watery mixture of components while stirring.

The invention thus also includes a process for the preparation of a composition of the invention comprising dissolution or mixing of the non-watery components into or with each other, and addition of the resultant oil phase to the watery mixture of components under stirring.

The following Examples illustrate the invention. The compounds are in free, i.e. neutral or base form unless specified otherwise.

### EXAMPLE 1

### Cosmeceutical Composition (Comprising Unsaturated Fatty Alcohol)

An oil-in-water emulsion is prepared containing the following excipients:

| | |
|---|---|
| Propyleneglycol | 5.0% |
| water | to 100% |
| oleyl alcohol | 10% |

further excipients:

| | |
|---|---|
| medium-chain triglycerides | 15% |
| cetyl alcohol | 4.0% |
| stearyl alcohol | 4.0% |
| sodium cetylstearyl sulfate | 1.0% |
| glycerine mono-/distearate | 2.0% |
| benzyl alcohol | 1.0% |
| citric acid 0.05%/sodium hydroxide | |
| 0.02% buffer to bring pH to 5.5 | |

The composition is prepared by mixing together the oleyl alcohol, the triglycerides, the propyleneglycol, the cetyl alcohol and the stearyl alcohol and heating to 65° C. until all components are dissolved and mixed. The sodium cetylstearyl sulfate and glycerine mono-/distearate are then added to the oil phase and stirred until all components are dissolved. The water is then heated separately in a vessel containing a stirrer and homogeneizer. The benzyl alcohol is added thereto and the oil phase is then slowly added to the watery mixture while stirring and homogenizing until a homogenous emulsion with a droplet size of less than 20 μm is obtained. The emulsion is then cooled to room temperature and the pH brought to 5.5 with the citrate buffer.

The emulsion is stable.

### EXAMPLE 2

### Cosmeceutical Composition (Devoid of Unsaturated Fatty Alcohol)

An oil-in-water emulsion is prepared containing the following excipients:

| | |
|---|---|
| Propyleneglycol | 5.0% |
| water | to 100% |

further excipients:

| | |
|---|---|
| medium-chain triglycerides | 15% |
| cetyl alcohol | 4.0% |
| stearyl alcohol | 4.0% |
| sodium cetylstearyl sulfate | 1.0% |
| glycerine mono-/distearate | 2.0% |
| benzyl alcohol | 1.0% |

The composition is prepared by mixing together the triglycerides, the propyleneglycol, the cetyl alcohol and the stearyl alcohol and heating to 65° C. until all components are dissolved and mixed. The sodium cetylstearyl sulfate and glycerine mono-/distearate are then added to the oil phase and stirred until all components are dissolved. The water is then heated separately in a vessel containing a stirrer and homogeneizer. The benzyl alcohol is added thereto and the oil phase is then slowly added to the watery mixture while stirring and homogenizing until a homogenous emulsion with a droplet size of less than 20 μm is obtained. The emulsion is then cooled to room temperature.

The emulsion is stable.

### EXAMPLE 3

### Cosmeceutical Composition (Comprising Unsaturated Fatty Alcohol)

An oil-in-water emulsion is prepared containing the following excipients:

| | |
|---|---|
| Propyleneglycol | 5.0% |
| water | to 100% |
| oleyl alcohol | 10% |

further excipients:

| | |
|---|---|
| medium-chain triglycerides | 15% |
| cetyl alcohol | 4.0% |
| stearyl alcohol | 4.0% |
| polysorbate 60 | 5.0% |
| sorbitan monostearate | 3.0% |
| methylparaben | 0.07% |
| propylparaben | 0.03% |

The composition is prepared by mixing together the oleyl alcohol, the triglycerides, the propyleneglycol, the cetyl alcohol, the stearyl alcohol, the polysorbate 60, the sorbitan monostearate and the parabens and heating to 65° C. until all components are dissolved and mixed. The water is then heated separately in a vessel containing a stirrer and homogeneizer. The oil phase is then slowly added to the watery mixture while stirring and homogenizing until a homogenous emulsion with a droplet size of less than 20 μm is obtained. The emulsion is then cooled to room temperature.

The emulsion is stable.

### EXAMPLE 4

### Topical Composition (For Infants and Babies)

A composition particularly suited for topical application to infants and babies is prepared from the following:

| Amount | |
|---|---|
| (wt. % based on | |
| total composition) | Excipient |
| 59.92 | purified water |
| 7.0 | dimethicone |
| 1.0 | sunflower oil |
| 14 | capric/caprylic triglycerides |
| 3.0 | propylene glycol |
| 3.0 | glycerine |
| 2.0 | glyceryl monostearate, SE |
| 4.0 to 6.0 | cetyl alcohol |
| 2.0 to 4.0 | stearyl alcohol |
| 1.0 | benzyl alcohol |
| 1.0 | sodium stearyl lactylate |
| 0.05 | tocopheryl acetate |
| 0.01 | Calendula extract |
| 0.02 | sodium hydroxide |
| 100 | q.s. w/water to |

The propylene glycol, water and glycerine are provided to a stainless steel jacketed tank with variable turbine mixing speeds and side sweep capabilities, and heated to 75-80° C. with moderate mixing until uniform to form a first phase (“phase A”).

To a separate tank equipped with variable speed mixer, are added dimethicone (Dow Corning 200® Fluid, 50 Cst), sunflower oil (Lipovol® SUN, Lipo Chemicals Inc.), capric/caprylic triglycerides Captex R355 (Abitec Corp.) cetyl alcohol, glyceryl monostearate, sodium stearyl lactylate (Capmul S18L^{R}, Abitec Corp.) and stearyl alcohol (Lipocol® S-20). The mixture is heated to 75-80° C. with mixing, to form a uniform second phase (“phase B”).

Phase B is combined with phase A, with increased agitation as well as side sweep. Mixing is continued until the combined phases appears homogeneous and smooth. The mixing speed is then reduced, and the mixture allowed to cool to 40° C. Benzyl alcohol is added with mixing to form a uniform emulsion. Tocopheryl acetate and Calendula extract are then added, with continued mixing.

The emulsion is cooled to room temperature and the pH brought to 5.5 with 50% sodium hydroxide or citric acid.

The resulting oil-in-water emulsion is stable.

The above composition has a low oily after-feel with good application aesthetics and quick rub-in. It is suitable for treating dry skin, diaper rash and scrapes.

1. A topical composition comprising:
a physiologically acceptable alkanediol, ether diol or diether alcohol containing up to 8 carbon atoms;
water; and
optionally an unsaturated fatty alcohol; and
optionally further conventional excipients, for use as a cosmeceutical.

2. A composition according to claim 1 comprising:
propyleneglycol;
water; and
optionally oleyl alcohol; and
further excipients, namely
medium-chain triglycerides;
cetyl alcohol;
stearyl alcohol;
sodium cetylstearyl sulfate or polysorbate 60 (Tween 60^{R});
glycerine mono/distearate or sorbitan monostearate (Arlacel 60^{R});
benzyl alcohol or methyl- and/or propylparaben; and
optionally citric acid/sodium hydroxide (buffer);
and optionally further conventional excipients.

3. A composition according to claim 1 comprising:
propyleneglycol from about 1% to about 20%;
water from about 25% to about 75%; and
optionally oleyl alcohol from about 5% to about 20%; and
further excipients, namely
medium-chain triglycerides from about 5% to about 50%;
cetyl alcohol from about 2% to about 10%;
stearyl alcohol from about 2% to about 10%;
sodium cetylstearyl sulfate or polysorbate 60 from about 0.5% to about 6%;
glycerine mono/distearate or sorbitan monostearate from about 1% to about 5%;
benzyl alcohol or methyl-/propylparaben from about 0.05% to about 2%; and
optionally citric acid/sodium hydroxide (buffer) to bring the pH to about 5.5;
and optionally further conventional excipients.

4. A process for the preparation of a composition according to any one of claims 1 to 3 comprising dissolution or mixing of the non-watery components into or with each other, and addition of the resultant oil phase to the watery mixture of components under stirring.

5. Use as a cosmeceutical of a composition according to any one of claims 1 to 3.

6. Use as a cosmeceutical according to claim 5 of a composition according to any one of claims 1 to 3 wherein the composition is a cream emulsion comprising:

| | wt. % |
|---|---|
| propyleneglycol | 5.0 |
| water | ad 100 |
| oleyl alcohol | 10 |
| medium chain triglycerides | 15 |
| cetyl alcohol | 4.0 |
| stearyl alcohol | 4.0 |
| sodium cetylstearyl sulfate | 1.0 |
| glycerine mono-/distearate | 2.0 |
| benzyl alcohol | 1.0 |
| citric acid | 0.05 |
| sodium hydroxide | 0.02 |

7. Use as a cosmeceutical of a composition according to any one of claims 1 to 3 and additionally comprising a pharmaceutically active agent.

8. Use as a cosmeceutical of a composition according to claim 7 wherein the pharmaceutically active agent is pimecrolimus.

9. Use as a cosmeceutical according to claim 8 wherein the additional pharmaceutically active agent is pimecrolimus 1% w/w and the composition is a cream emulsion comprising:

| | wt. % |
|---|---|
| propyleneglycol | 5.0 |
| water | ad 100 |
| oleyl alcohol | 10 |
| medium chain triglycerides | 15 |
| cetyl alcohol | 4.0 |
| stearyl alcohol | 4.0 |
| sodium cetylstearyl sulfate | 1.0 |
| glycerine mono-/distearate | 2.0 |
| benzyl alcohol | 1.0 |
| citric acid | 0.05 |
| sodium hydroxide | 0.02 |

10. Use of a composition according to any one of claims 1 to 3 in the preparation of a cosmeceutical composition.

11. A method of repairing or maintaining skin barrier function comprising administering a composition according to any one of claims 1 to 3 to a subject in need thereof.

12. A topical composition comprising:
at least one pharmaceutically active agent
a physiologically acceptable alkanediol, ether diol or diether alcohol containing up to 8 carbon atoms;
water; and
optionally an unsaturated fatty alcohol; and
optionally further conventional excipients,
provided that said at least one pharmaceutically active agent is other than a pharmaceutically active macrolide compound of the FK 506 class.

13. A topical composition comprising:
a physiologically acceptable alkanediol, ether diol or diether alcohol containing up to 8 carbon atoms;
water; and
optionally an unsaturated fatty alcohol; and
optionally further conventional excipients, for use as an infant and baby lotion.

14. A topical composition according to claim 13 for use as an infant and baby lotion additionally comprising:
a liquid oil component selected from one or more of dimethicone, sunflower oil and capric/caprylic triglycerides
an emulsifier selected from one or more of glyceryl monostearate, cetyl alcohol, stearyl alcohol, and sodium stearyl lactylate.

15. A composition suitable for topical application to infants and babies comprising:

| Amount | |
|---|---|
| (wt. % based on | |
| total composition) | Excipient |
| 50-70 | purified water |
| 5-10 | dimethicone |
| 1-5 | sunflower oil |
| 1-15 | capric/caprylic triglycerides |
| 1-10 | propylene glycol |
| 1-5 | glyceryl monostearate, SE |
| 2-10 | cetyl alcohol |
| 1-5 | stearyl alcohol |
| 1-5 | sodium stearyl lactylate |
| q.s. | e.g., sodium hydroxide, to bring |
| | pH to 5.5 |

16. A composition according to claim 15 additionally comprising 0.01-0.1 wt. % tocopheryl acetate and 0.005-0.05 wt. % Calendula extract.

17. A composition according to claim 16 additionally comprising glycerine, benzyl alcohol and sodium hydroxide.
